(19) United States
(12) Patent Application Publication
(10) Pub. No.: US 2024/0207765 A1
(43) Pub. Date: Jun. 27, 2024

(54) VIRAL FILTRATION MEDIA, ARTICLES, AND METHODS

(71) Applicant: 3M INNOVATIVE PROPERTIES COMPANY, St. Paul, MN (US)

(72) Inventors: Rebecca A. HOCHSTEIN, St. Paul, MN (US); Alexei M. VOLOSHIN, Woodbury, MN (US); Jonathan F. HESTER, Hudson, WI (US); Daniel J. O'NEAL, St. Paul, MN (US); Minghua DAI, Plymouth, MN (US)

(21) Appl. No.: 18/555,612

(22) PCT Filed: Apr. 28, 2022
(86) PCT No.: PCT/IB2022/053930

§ 371 Date: Oct. 16, 2023

Related U.S. Application Data
(60) Provisional application No. 63/182,236, filed on Apr. 30, 2021.

(51) Int. Cl. B01D 39/16; B01D 39/02; B01D 39/08; B01D 39/20; B01D 67/00; B01D 71/40; B01D 71/52; B01D 71/78

(52) U.S. Cl. CPC B01D 39/1623; B01D 39/02; B01D 39/08; B01D 39/1676; B01D 39/1692; B01D 39/202; B01D 39/2024; B01D 39/2027; B01D 39/2068; B01D 67/00931; B01D 71/401; B01D 71/52; B01D 71/78; B01D 2239/0618; B01D 2239/0622; B01D 2239/0627; B01D 2239/0636; B01D 2239/0654; B01D 2239/1216; B01D 2239/1233; B01D 2323/38

(57) ABSTRACT

Viral filtration media, an article comprising the viral filtration media, and a method of filtering a virus-containing sample using the viral filtration media, wherein the viral filtration media comprises: a porous substrate comprising a surface having a polymer grafted thereto, wherein the grafted polymer comprises interpolymerized monomers comprising: a (meth)acrylic acid monomer: and, optionally, a poly (alkylene oxide) monomer.

### BACKGROUND

Detection, quantification, isolation, and purification of target biomaterials, particularly viruses and viral vectors, have long been objectives of investigators. Detection and quantification are important diagnostically, for example, as indicators of various physiological conditions such as diseases. Isolation and purification are important for therapeutic uses and in biomedical research.

Polymeric materials have been widely used for the separation and purification of viruses and viral vectors. Such separation and purification methods can be based on any of a number of binding factors or mechanisms including the presence of an ionic group, the size of the target virus, a hydrophobic interaction, an affinity interaction, the formation of a covalent bond, and so forth. New materials having new mechanisms for interaction with viruses are desired.

### SUMMARY OF THE DISCLOSURE

The present disclosure provides viral filtration media, an article comprising the viral filtration media, and a method of filtering a virus-containing sample using the viral filtration media.

In one embodiment, the present disclosure provides viral filtration media comprising: a porous substrate comprising a surface having a polymer grafted thereto, wherein the grafted polymer comprises interpolymerized monomers comprising: a (meth)acrylic acid monomer; and a poly(alkylene oxide) monomer optionally including a hydrocarbon chain.

In another embodiment, the present disclosure provides an article comprising the viral filtration media described herein.

In another embodiment, the present disclosure provides a method of filtering a virus-containing sample, the method comprising: providing a viral filtration media comprising: a porous substrate comprising a surface having a polymer grafted thereto, wherein the grafted polymer comprises interpolymerized monomers comprising: a (meth)acrylic acid monomer: and, optionally, a poly(alkylene oxide) monomer (optionally including a hydrocarbon chain): providing a virus-containing sample comprising a target virus: and contacting the viral filtration media with the virus-containing sample under conditions effective to separate the target virus from other material in the sample.

As used herein, “alkyl” refers to a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 40 carbon atoms. In some embodiments, the alkyl groups contain 1 to 30 carbon atoms, 1 to 20, 1 to 12 carbon atoms, 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkylene” refers to a divalent group that is a radical of an alkane and includes groups that are linear, branched, cyclic, bicyclic, or a combination thereof. Unless otherwise indicated, the alkylene group typically has 1 to 40 carbon atoms. In some embodiments, the alkylene group has 1 to 30 carbon atoms, 1 to 20 carbon atoms, 1 to 12 carbon atoms, 1 to 10 carbon atoms, 1 to 6 carbon atoms, or 1 to 4 carbon atoms. In some embodiments, the alkylene is a linear saturated divalent hydrocarbon having from 1 to 12 carbon atoms, and in some embodiments, the alkylene is a branched saturated divalent hydrocarbon having from 3 to 12 carbon atoms, e.g., methylene, ethylene, propylene, 2-methylpropylene, pentylene, hexylene, 1,4-cyclohexylene, 1,4-cyclohexyldimethylene, and the like.

The term “alkylene oxide” refers to a divalent group that is an oxy group bonded directly to an alkylone group.

The term “ethylenically unsaturated group” refers to those groups having carbon-carbon double (or triple) bonds that may be free-radically polymerized, and includes (meth)acrylamides, (meth)acrylates, vinyl and vinyloxy groups, allyl and allyloxy groups, and acetylenic groups.

The term “(meth)acrylic acid” includes acrylic acid and methacrylic acid. Similarly, the term “(meth)acrylamide” includes acrylamide and methacrylamide.

The terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other claims may also be preferred, under the same or other circumstances. Furthermore, the recitation of one or more preferred claims does not imply that other claims are not useful and is not intended to exclude other claims from the scope of the disclosure.

In this application, terms such as “a,” “an,” and “the” are not intended to refer to only a singular entity, but to include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the term “room temperature” refers to a temperature of 20° C. to 25° C. or 22ºC to 25° C.

The term “in the range” or “within a range” (and similar statements) includes the endpoints of the stated range.

Groupings of alternative elements or embodiments disclosed herein are not to be construed as limitations. Each group member may be referred to and claimed individually or in any combination with other members of the group or other elements found therein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF THE FIGURES

FIG. 1 is a schematic of a random copolymer of methacrylic acid and a poly(alkylene oxide) methacrylate, wherein “x” and “y” represent the amount of methacrylic acid and poly(alkylene oxide) methacrylate monomeric units, respectively, “m” represents the number of ethylene oxide repeat units, and “n” represents the number of methylene groups.

FIG. 2 shows a perspective view of a filter capsule including a media stack that includes the filtration media described herein.

### DETAILED DESCRIPTION OF ILLUSTRATIVE EMBODIMENTS

The present disclosure provides viral filtration media, an article comprising the viral filtration media, and a method of filtering a virus-containing sample using the viral filtration media.

The viral filtration media (i.e., filter media) includes: a porous substrate comprising a surface having a polymer grafted thereto, wherein the grafted polymer comprises interpolymerized monomers comprising: a (meth)acrylic acid monomer; and, in certain embodiments, a poly(alkylene oxide) monomer optionally including a hydrocarbon chain. Alternatively stated, the viral filtration media includes poly(meth)acrylic acid and, optionally, poly(alkylene oxide) grafted onto a porous substrate.

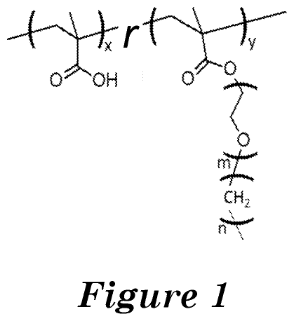

An exemplary grafted polymer is disclosed in FIG. 1, which shows a schematic of a random copolymer of methacrylic acid and a poly(alkylene oxide) methacrylate. Pegylation, e.g., the addition of poly(ethylene glycol) (PEG) or poly(ethylene glycol) behenyl ether chains, for example, to (meth)acrylic acid grafted to a porous substrate (e.g., a polypropylene nonwoven substrate) provides a method of shielding or reducing the poly(meth)acrylic acid-functionality of the substrate. That is, the addition of poly(alkylene oxide) chains to functional nonwovens provides the ability to shield or tune the functional chemistry (i.e., the poly(meth)acrylic acid), allowing for the ability to shield or lessen the effect of the functional chemistry and potentially change how strongly it interacts with virus. This is especially useful for virus purification, where the target virus particles need to be separated from cells and cell debris, DNA, host cell proteins, and/or other virus particles. For example, for Lentivirus (i.e., a virus-like particle having a lipid envelope made out of the same lipid as its mammalian cell host, which is used as a viral vector in gene therapy) clarification, adding polyethylene glycol chains to a methacrylic acid functionalized nonwoven enables the trapping of cells and cell debris while allowing the Lentivirus particles to flow through.

The (meth)acrylic acid (as opposed to a (meth)acrylate) is believed to remove virus by binding thereto, which is surprising because virus has a net negative charge. The poly(alkylene oxide) is believed to modulate the separation of one virus from another by tailoring the interaction with viruses. For example, addition to the grafted polymer of poly(alkylene oxide) groups and, optionally, alkyl groups (R² groups in Formula (I) below) enables the separation of enveloped viruses from nonenveloped viruses.

In certain embodiments, the (meth)acrylic acid monomer is methacrylic acid (H₂C═CH(CH₃)C(═O)OH). Esters of the (meth)acrylic acid monomers are not believed to be as effective at binding viruses.

In certain embodiments, the (meth)acrylic acid monomer (“x” in FIG. 1) is present in an amount of at least 25 wt-%, or at least 50 wt-%, based on the total weight of the interpolymerized monomers. In certain embodiments, the (meth)acrylic acid monomer is present in an amount of up to 98 wt-%, or up to 95 wt-%, based on the total weight of the interpolymerized monomers. It is believed that such high concentrations of (meth)acrylic acid monomers enhances the binding of viruses.

The poly(alkylene oxide) group of the poly(alkylene oxide) monomer may be a homopolymer (e.g., poly(ethylene oxide) or poly(propylene oxide)) or copolymer (e.g., poly(ethylene-co-propylene oxide)). The copolymer may be a block, random, or gradient copolymer.

In certain embodiments, the poly(alkylene oxide) monomer used is a poly(ethylene glycol) methyl ether methacrylate or a poly(ethylene glycol) methacrylate represented by the abbreviation of PEGMA. In certain embodiments, the poly(alkylene oxide) monomer is a poly(ethylene glycol) methyl ether methacrylate having a number-average molecular weight of about 300 g/mol (PEGMA-300), a poly(ethylene glycol) methacrylate having a number-average molecular weight of about 360 g/mol (PEGMA-360), a poly(ethylene glycol) methyl ether methacrylate having a number-average molecular weight of about 950 g/mol (PEGMA-950), or a poly(ethylene glycol) methyl ether methacrylate having a number-average molecular weight of about 2000 g/mol (PEGMA-2000).

In certain embodiments, the poly(alkylene oxide) monomer is a poly(ethylene glycol) methyl ether methacrylate having a number-average molecular weight between 160 g/mol and 5000 g/mol. In certain embodiments, the poly(alkylene oxide) monomer is a poly(ethylene glycol) methyl ether methacrylate having a number-average molecular weight between 160 g/mol and 3000 g/mol. In certain embodiments, the poly(alkylene oxide) monomer is a poly(ethylene glycol) methyl ether methacrylate having a number-average molecular weight between 160 g/mol and 2000 g/mol.

In certain embodiments, the poly(alkylene oxide) monomer has the formula:

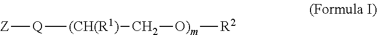

wherein: Z is a polymerizable ethylenically unsaturated group; Q is a divalent linking group; R¹ is H or an alkyl group; R² is H or an alkyl group; and m is 2 to 100.

In certain embodiments, Z is selected from the group consisting of:

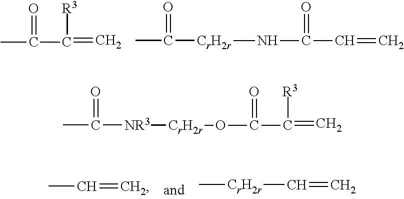

wherein: R³ is H or CH₃ (preferably, R³ is CH₃); and r is 1-10.

In certain embodiments, Q is selected from the group of —O—, —NR³—, —C(O)O—, and —C(O)NR³—, wherein R³ is H or CH₃ (preferably, R³ is H). In certain embodiments, Q is selected from the group of —O— and —NR³—, wherein R³ is H or CH₃ (preferably, R³ is H). In certain embodiments, Q is —O—.

In certain embodiments, the poly(alkylene oxide) monomer comprises a poly(alkylene oxide) (meth)acrylate monomer (wherein Z is —C(O)—C(R³)—CH₂).

In certain embodiments, R¹ is H or a C1-C4 alkyl group. In certain embodiments, R¹ is H.

In certain embodiments, R² is H or a C1-C40 alkyl group. In certain embodiments, R² is a C1-C40 alkyl group (i.e., the poly(alkylene oxide) monomer includes a hydrocarbon chain of at least 1 carbon and up to 40 carbons).

In certain embodiments, m is 2 to 50.

The poly(alkylene oxide) monomer (i.e., the monomer having a poly(alkylene oxide) group) can be prepared, for example, by reacting mono- or di-functional alkylene oxide (co)polymers (which are typically commercially available) with reactive ethylenically unsaturated compounds (e.g., acrylates). The functional groups terminating the poly(alkylene oxide) may include hydroxy groups, amine groups, and carboxy groups. A variety of reactive ethylenically unsaturated compounds such as acrylate derivatives can be used including, but not limited to, (meth)acrylic acid, (meth)acryloyl chloride, (meth)acrylic anhydride, and 2-isocyanatoethyl (meth)acrylate. Preferably, the monomer is prepared by reacting the mono- or di-functional alkylene oxide (co)polymer with (meth)acrylic anhydride. Typically, if a stoichiometric amount of the ethylenically unsaturated reactant is combined with the monofunctional alkylene oxide (co)polymer (such as a monohydroxy terminated alkylene oxide (co)polymer), 100% conversion to the monosubstituted product is obtained.

Examples of suitable monofunctional poly (alkylene oxide) monomers include poly(ethylene oxide) (meth)acrylate, poly (propylene oxide) (methacrylate, polyethylene oxide-propylene oxide) (meth)acrylate, poly(ethylene glycol) (meth)acrylate, poly(ethylene glycol) methyl ether (meth)acrylate, poly(ethylene glycol) behenyl ether (meth)acrylate, and combinations thereof. Such monomers preferably include one nonreactive end group such as C1-C4 alkoxy, aryloxy (e.g., phenoxy), and C1-C4 alkaryloxy. These groups can be linear or branched. These monomers can be of a wide range of molecular weights and are commercially available from sources such as Sartomer Company, Exton, PA: Shinnakamura Chemical Co., Ltd., Tokyo, Japan; MilliporeSigma, Milwaukee. WI: and Osaka Organic Chemical Ind., Ltd., Osaka, Japan.

In certain embodiments, the poly(alkylene oxide) monomer is present in an amount of at least 2 wt-%, or at least 5 wt-%, based on the total weight of the interpolymerized monomers. In certain embodiments, the poly(alkylene oxide) monomer is present in an amount of up to 75 wt-%, or up to 50 wt-%, based on the total weight of the interpolymerized monomers.

In certain embodiments, the PEG component comprises at least 10 wt-%, at least 15 wt-% or at least 20 wt-% of the grafted polymer (calculated as described below in Example 18). In certain embodiments, the PEG component comprises up to 80 wt-%, up to 60 wt-%, or up to 40 wt-% of the grafted polymer (calculated as described below in Example 18).

### Substrates

The polymers described herein may be grafted to a porous substrate to form filter media that can be used in filter elements.

The porous substrate can be in essentially any form such as particles, fibers, films, webs, membranes, sponges, or sheets. Suitable porous substrates can be organic, inorganic, or a combination thereof (preferably, organic: more preferably, polymeric). Suitable porous substrates include porous particles, porous membranes, porous nonwoven webs, porous woven webs, porous sponges, porous fibers, and the like, and combinations thereof.

In certain embodiments, porous substrates include porous nonfibrous membranes as well as porous nonwoven webs and other porous fibrous substrates. Although in certain embodiments, the substrate is not a membrane.

In certain preferred embodiments, the porous substrate is a porous nonwoven web. As used herein, the terms “nonwoven web” or “nonwoven substrate” are used interchangeably and refer to a fabric that has a structure of individual fibers or filaments which are randomly and/or unidirectionally interlaid in a mat-like fashion.

A fibrous nonwoven web can be made by carded, air laid, spunlaced, hydroentangled, spunbonding, or melt-blowing techniques, or combinations thereof. Spunbonded fibers are typically small diameter fibers that are formed by extruding molten thermoplastic polymers as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g., air) stream which attenuates the filaments of molten thermoplastic material to reduce their diameter. Thereafter, the meltblown fibers are carried by the high velocity gas stream and are deposited on a collecting surface to from a web of randomly disbursed meltblown fibers. Any of the nonwoven webs may be made from a single type of fiber or two or more fibers that differ in the type of thermoplastic polymer and/or thickness.

Suitable nonwoven substrates may have a tensile strength of at least 4.0 newtons prior to grafting, a surface area of 15 m² per square meter (m²/m²) to 50 m²/m² of nonwoven substrate, a mean pore size of 1 micron to 40 microns according to ASTM F 316-03, and a solidity of less than 20%.

The fibers of the nonwoven substrate typically have an effective fiber diameter (EFD) of from 3 to 20 micrometers, preferably from 4 to 10 micrometers, as calculated according to the method set forth in Davies. C N., “The Separation of Airborne Dust and Particles,” Institution of Mechanical Engineers. London, Proceedings 1B, 1952. The nonwoven substrate preferably has a basis weight in the range of 10 to 400 g/m², more preferably 100 to 300 g/m². The average thickness of the nonwoven substrate is preferably 0.1 to 10 mm, more preferably 0.25 to 5 mm for the non-functionalized, uncalendared substrate. The minimum tensile strength of the nonwoven web is preferably 4.0 Newtons/cm. It is generally recognized that the tensile strength of nonwovens is lower in the machine direction than in the cross-web direction due to better fiber bonding and entanglement in the latter.

Nonwoven web loft is measured by solidity, a parameter that defines the solids fraction in a volume of web. Lower solidity values are indicative of greater web loft. Useful nonwoven substrates have a solidity of less than 20%, preferably less than 15%. Solidity is a unitless fraction typically represented by α: α=m₁÷(p_{f}×L_{nonweven}), wherein, m_{f} is the fiber mass per sample surface area: p_{f} is the fiber density: and L_{nonwoven} is the nonwoven thickness. Solidity is used herein to refer to the nonwoven substrate itself and not to the functionalized nonwoven. When a nonwoven substrate contains mixtures of two or more kinds of fibers, the individual solidifies are determined for each kind of fiber using the same L_{nonwoven} and these individual solidities are added together to obtain the web's solidity, α.

Exemplary manufacturing methods of non-woven webs may be found in Wente, Superfine Thermoplastic Fibers. 48 INDUS. ENG. CHEM. 1342(1956), or in Wente et al., Manufacture of Superfine Organic Fibers. (Naval Research Laboratories Report No 4364, 1954). Useful methods of preparing the nonwoven substrates are also described in U.S. RE39.399 (Allen). U.S. Pat. No. 3,849,241 (Butin et al.). U.S. Pat. No. 7,374,416 (Cook et al.), U.S. Pat. No. 4,936,934 (Buehning), and U.S. Pat. No. 6,230,776 (Choi).

The porous substrate may be formed from any suitable thermoplastic polymeric material. Suitable polymeric materials include, but are not limited to, polyolefins, poly(isoprenes), poly(butadienes), fluorinated polymers, chlorinated polymers, polyamides, polyimides, polyethers, poly(ether sulfones), poly(sulfones), poly(vinyl acetates), copolymers of vinyl acetate, such as poly(ethylene)-co-poly(vinyl alcohol), poly(phosphazenes), poly(vinyl esters), poly(vinyl ethers), poly(vinyl alcohols), and poly (carbonates).

Suitable polyolefins include, but are not limited to, poly(ethylene), poly (propylene), poly(l-butene), copolymers of ethylene and propylene, alpha olefin copolymers (such as copolymers of ethylene or propylene with 1-butene, 1-hexene, 1-octene, and 1-decene), poly(ethylene-co-1-butene) and poly(ethylene-co-1-butene-co-1-hexene). Suitable fluorinated polymers include, but are not limited to, poly(vinyl fluoride), poly(vinylidene fluoride), copolymers of vinylidene fluoride (such as poly(vinylidene fluoride-co-hexafluoropropylene), and copolymers of chlorotrifluoroethylene (such as poly(ethylene-co-chlorotrifluoroethylene).

Suitable polyamides include, but are not limited to, poly(iminoadipoyliminohexamethylene), poly(iminoadipoyliminodecamethylene), and polycaprolactam. Suitable polyimides include, but are not limited to, poly (pyromellitimide). Suitable poly(ether sulfones) include, but are not limited to, poly(diphenylether sulfone) and poly(diphenylsulfone-co-diphenylene oxide sulfone).

Suitable copolymers of vinyl acetate include, but are not limited to, poly(ethylene-co-vinyl acetate) and such copolymers in which at least some of the acetate groups have been hydrolyzed to afford various poly(vinyl alcohols).

In some embodiments, the porous substrate is formed from a propylene homo- or copolymers, most preferably propylene homopolymers. Polypropylene polymers are often a material of choice for porous articles, such as nonwovens and microporous films, due to properties such as non-toxicity, inertness, low cost, and the ease with which it can be extruded, molded, and formed into articles.

In certain embodiments, the porous substrate is a nonfibrous porous membrane. Examples may be formed from any suitable thermoplastic polymeric material as described above for the fibrous substrate.

In some embodiments, the nonfibrous porous membrane is a microporous membrane such as a thermally induced phase separation (TIPS) membrane. TIPS membranes are often prepared by forming a homogenous solution of a thermoplastic material and a second material above the melting point of the thermoplastic material. Upon cooling, the thermoplastic material crystallizes and phase separates from the second material. The crystallized thermoplastic material is often stretched. The second material is optionally removed either before or after stretching. Microporous membranes are further disclosed in U.S. Pat. No. 4,539,256 (Shipman), U.S. Pat. No. 4,726,989 (Mrozinski), U.S. Pat. No. 4,867,881 (Kinzer), U.S. Pat. No. 5,120,594 (Mrozinski), U.S. Pat. No. 5,260,360 (Mrozinski et al.), and U.S. Pat. No. 5,962,544 (Waller). Further, the microporous film can be prepared from ethylene-vinyl alcohol copolymers as described in U.S. Pat. No. 5,962,544 (Waller).

Some exemplary TIPS membranes include poly(vinylidene fluoride) (PVDF), polyolefins such as polyethylene homo- or copolymers or polypropylene homo- or copolymers, vinyl-containing polymers or copolymers such as ethylene-vinyl alcohol copolymers and butadiene-containing polymers or copolymers, and acrylate-containing polymers or copolymers. TIPS membranes comprising PVDF are further described in U.S. Pat. No. 7,338,692 (Smith et al.). In another exemplary embodiment, the porous nonfibrous membrane is a microporous membrane such as a solvent-induced phase separation (SIPS) membrane. SIPS membranes are often prepared by forming a homogenous solution of a thermoplastic material and a second material (a solvent), the solution is cast into a film or hollow fiber form, then immersed in a nonsolvent bath. The nonsolvent causes the thermoplastic material to solidify, or phase separate, and also extracts out the solvent, leaving a porous polymeric membrane. Examples of SIPS membranes prepared from polyamides include a nylon microporous film or sheet, such as those described in U.S. Pat. No. 6,056,529 (Meyering et al.), 6,267,916 (Meyering et al.), 6,413,070 (Meyering et al.), 6,776,940 (Meyering et al.), 3,876,738 (Marinacchio et al.), 3,928,517 (Knight et al.), 4,707,265 (Knight et al.), and 5,458,782 (Hou et al.). Other examples include microporous membranes prepared from polysulfones and polyethersulfones, many of which are commercially available from 3M Company, St. Paul, MN, e.g., MicroPES and DuraPES.

Polymers of the present disclosure may be disposed on (e.g., grafted to) a porous substrate using standard techniques, which are exemplified in the Examples Section.

### Methods of Making Filtration Media

Grafting of the monomers to the surface of the porous substrate preferably involves the use of a (meth)acrylate functional group due to relatively slow, more uniform reactivity and durability of such groups to a porous substrate that has been exposed to ionizing radiation (e.g., e-beam irradiation).

Functionalized substrates of the present disclosure may be prepared using above-described monomers to provide a grafted polymer on the surface of a porous (e.g., nonwoven) base substrate. When two or more of the above-described grafting monomers are used, the monomers may be grafted onto the porous base substrate in a single reaction step by exposing it to an ionizing radiation followed by contacting it with all grafting monomers present. Alternatively, the monomers may be grafted in sequential reaction steps by first exposing the substrate to ionizing radiation followed by contacting it with one or more grafting monomers, then a second exposure to an ionizing radiation and a second contact with grafting monomers after the second exposure to the ionizing radiation. In the contacting steps, typically a monomer solution is allowed to at least partially fill the void volume of the porous base substrate. This method is further described in U.S. Pat. No. 8,328,023 (Weiss et al.) and U.S. Pat. No. 10,352,835 (Rasmussen et al.).

### Articles

The filtration media of the present disclosure is particularly useful in an article for viral capture or viral purification, for example. Such articles include a porous polymeric substrate as described herein, and grafted to such porous substrate, a polymer of the present disclosure. Such substrates with the polymers described herein grafted thereto may serve as a filter element.

The grafted polymer can alter the original nature of the porous substrate. The resulting polymer-bearing porous substrates can retain many of the advantages of the original porous substrate (for example, mechanical and thermal stability, porosity, and so forth) but can also exhibit enhanced affinity for biomaterials such as viruses, proteins, and the like.

Articles comprising the polymer-bearing porous substrates can further include conventional components such as housings, holders, adapters, and the like, and combinations thereof.

If desired, efficiency of viral filtration can be improved by using a plurality of stacked or layered, functionalized porous substrates (for example, functionalized porous nonwoven webs) as a filter element. Thus, a filter element can comprise one or more layers of functionalized porous substrate. The individual layers of the filter element can be the same or different. The layers can vary in porosity, degree of grafting, and so forth. The filter element can further comprise an upstream prefilter layer and/or a downstream support layer. The individual layers can be planar or pleated, as desired.

Examples of suitable prefilter and support layer materials include any suitable porous membranes of polyethersulfone, polypropylene, polyester, polyamide, resin-bonded or binder-free fibers (for example, glass fibers), and other synthetics (woven and nonwoven fleece structures); sintered materials such as polyolefins, metals, and ceramics: yarns: special filter papers (for example, mixtures of fibers, cellulose, polyolefins, and binders): polymer membranes: and the like; and combinations thereof.

Useful articles for viral filtration include a filter cartridge including one or more of the above-described filter elements, a filter assembly comprising one or more of the above-described filter elements and a filter housing, and the like. The articles can be used in carrying out a method of the present disclosure, which may involve, for example, capture of a virus or purification of a virus.

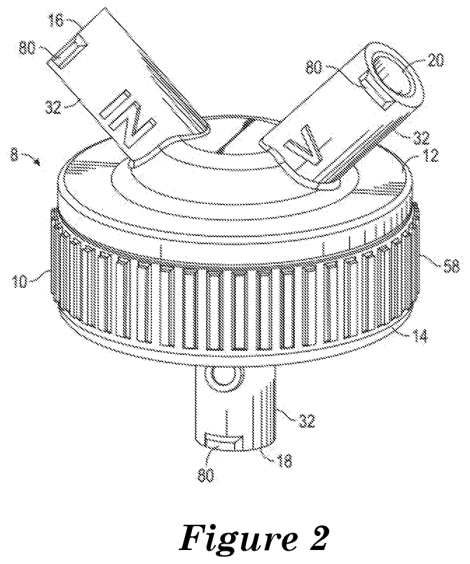

FIG. 2 shows a perspective view of a filter capsule having a housing with an inlet, an outlet, an optional vent, and a media stack (not shown) positioned between the inlet and the outlet to purify a virus-containing sample. The media stack includes the filtration media described herein.

The housing can be any suitable size, with the size scaled as appropriate to the media surface area within the housing. Typically, laboratory scale devices will be relatively small and have low hold-up volumes for processing limited amounts of fluid. Pilot scale and production scale devices will have corresponding larger amounts of media within them to process larger amounts of fluid for each run. For example, laboratory scale devices may have media surfaces areas from 1 square centimeter (cm²) to 25 cm², pilot scale devices from 170 cm² to 1020 cm², and production scale devices from 2300 cm² to 16,100 cm². Other housing sizes and media volumes can be provided as needed for the specific application. Suitable housings are made by 3M Company, St. Paul, MN, and used in the 3M EMPHAZE AEX Hybrid Purifier product line. See https://www.3m.com/3M/en_US/company-us/all-3m-products/˜/3M-Emphaze-AEX-Hybrid-Purifier/?N=5002385+3291555558&rt=rud. Similar size housings and designs can be used to contain the media stack of this disclosure.

A suitable filtration device is disclosed in WO 2020/148,607 (3M Company). As shown in FIG. 2 in the device 8, a housing 10 is formed by joining an upper housing 12 to a lower housing 14. The housing 10 has an inlet 16, an outlet 18, and an optional vent 20. Disposed between the inlet 16 and the outlet 18 is a media stack (not specifically shown) within housing 10 in a chamber such that fluid from the inlet 16 enters the internal chamber and then passes through the media stack and out the outlet 18. The chamber is in fluid communication with the inlet 16 and the vent 20 such that any air in the chamber can be purged out the vent 20. A Luer lock connector (not shown) can be attached to the vent 20 and used as a valve to purge the air from the chamber until liquid from the inlet 16 begins to exit from the vent 20 and the valve is closed. Cylindrical projections 32 with opposing transverse tabs 80 extend from the housing and have a tapered bore to attach the Luer Lock connectors to the inlet, the outlet, and the vent. Longitudinal ribs 58 are spaced along the perimeter to provide enhanced grip while handling the housing.

### Methods of Filtering

Methods of the present disclosure include filtering a virus-containing sample (i.e., a sample including a target virus). Exemplary virus-containing samples include a biopharmaceutical drug, protein product or viral vector product, cell culture fluid containing a biopharmaceutical drug, protein product, or a viral vector product, clarified cell culture fluid containing a biopharmaceutical drug, protein product, or a viral vector product.

Target viruses include both enveloped viruses and non-enveloped viruses. Enveloped viruses are strongly negatively charged and adsorb to most depth filters. Exemplary target viruses include enveloped viruses such as Lentivirus, HIV-1 XMuLV, EMuLV, Measles, influenza, Herpes Simplex virus, and BVDV. Exemplary target viruses and viral vectors include non-enveloped viruses such as Adeno-associated virus, Adenovirus, Minute Virus of Mouse, Reovirus 3, PPV, and SV40.

The methods include: providing a viral filtration media as described herein, which may be incorporated in a filter element: providing a virus-containing sample comprising a target virus: and contacting the viral filtration media with the virus-containing sample under conditions effective to separate the target virus from other material in the sample. Herein, the “target” virus may be the desired material for further manipulation, or not. That is, the “other material” may be the desired material for further manipulation.

Herein, “conditions effective” include time the sample is in contact with the media, pH, conductivity, and combinations thereof.

In this context, “filtering” a virus-containing sample means separating a target virus from other material in a sample. This encompasses removing a target virus from a sample, e.g., by binding the target virus to the viral filtration media. It also includes binding other material in the sample and allowing the target virus to remain in the sample (e.g., that flows through the filtration media). This can include separating one virus from another virus (e.g., enveloped virus from non-enveloped virus).

In this context, “other material in the sample” can include biomaterials or biological species other than viruses (including relatively neutral or charged biomaterials such as non-viral microorganisms, acidic carbohydrates, proteins, nucleic acids, endotoxins, bacteria, cells, cellular debris, and the like).

The filtration media of the present disclosure can be used for viral clearance or viral purification. Virus purification involves removal of impurities (e.g., adventitious viruses) from a virus or viral vector, which is the product. Viral clearance involves the removal of viruses from a biopharma product. This is often referred to in terms of log reduction value (LRV), or the number of logs of virus that is removed from the sample. Virus is added to the sample to demonstrate the virus removal capabilities. Often 4 LRV is a desired level to achieve in order for the viral clearance to be considered robust (4 LRV=removal of 99.99% of a virus).

In certain embodiments, “contacting the viral filtration media with the virus-containing sample” includes allowing a moving virus-containing sample to impinge upon an upstream surface of the filter element (or the filtration media) for a time sufficient to effect separation of at least a portion of the target virus from other material in the virus-containing sample. Separation may result by binding the target virus to the filtration media and allowing the other material to flow through. Alternatively, separation may result by binding the other material to the filtration media and allowing the target virus to flow through.

In certain embodiments if the virus binds to the filtration media, at least a portion of the target virus means at least 3 LRV (99.9%), at least 4 LRV, or at least 6 LRV (99.9999%) of the target virus in the virus-containing sample is bound to the filter media in the filter element. In certain embodiments wherein the virus is the flow-through product, at least a portion of the target virus means at least 30%, at least 40%, at least 50%, at least 60%, at least 70%, at least 80%, at least 90%, or at least 100%, of the target virus is separated from other material in the virus-containing sample.

In certain embodiments, “contacting the viral filtration media with the virus-containing sample” includes allowing a moving virus-containing sample to impinge upon an upstream surface of the filter element (or the filtration media) for a time sufficient to effect binding of at least a portion of the target virus in the virus-containing sample to the filtration media. In certain embodiments, at least a portion of the target virus means at 3 LRV (99.9%), at least 4 LRV, or at least 6 LRV (99.9999%) of the target virus in the virus-containing sample is bound to the filter media in the filter element. The “other material” in the sample may be the desired product for further manipulation.

In a specific example, the media of the present disclosure can be used for removal of enveloped virus from a monoclonal antibody product. For example, the filtration media can be used to remove enveloped viruses from a sample and allow the monoclonal antibody product to flow through.

In another specific example, the media of the present disclosure can be used for removal of virus from a nucleic acid product. For example, the filtration media can be used to remove viruses from a sample and allow the nucleic acid product to flow through. In certain embodiments, filtration media that includes homopolymerized (meth)acrylic acid grafted onto a porous substrate removes all viruses. In certain embodiments, filtration medial that includes polymerized (meth)acrylic acid and polyethylene glycol pendent groups grafted onto a porous substrate removes primarily only enveloped viruses.

In certain embodiments, “contacting the viral filtration media with the virus-containing sample” includes allowing a moving virus-containing sample to impinge upon an upstream surface of the filter element (or the filtration media) for a time sufficient to effect binding of at least a portion of other material in the virus-containing sample to the filtration media. In certain embodiments, at least a portion of the other material means at least 30%, at least 40%, at least 50%, at least 60%, at least 70%, at least 80%, at least 90%, or at least 100%, of the other material in the virus-containing sample is bound to the filter media in the filter element.

In a specific example, the media of the present disclosure can be used for clarification of a virus product. For example, the filtration media can be used to remove cells and cell debris from a lentivirus culture and allow the lentivirus to flow through.

In certain embodiments, “contacting the viral filtration media with the virus-containing sample” includes allowing a moving virus-containing sample to impinge upon an upstream surface of the filter element for a time sufficient to effect separation of at least a portion of the target virus from another virus in the virus-containing sample. In certain embodiments, at least a portion of the target virus means at least 30%, at least 40%, at least 50%, at least 60%, at least 70%, at least 80%, at least 90%, or at least 100%, of the target virus in the virus-containing sample is separated from another virus in the virus-containing sample.

In a specific example, the media of the present disclosure can be used for removal of an enveloped virus from a nonenveloped virus product. For example, the filtration media can be used to remove enveloped viruses from a sample and allow nonenveloped viruses to flow through.

### EXEMPLARY EMBODIMENTS

Embodiment 1 is a viral filtration media comprising: a porous substrate comprising a surface having a polymer grafted thereto, wherein the grafted polymer comprises interpolymerized monomers comprising: a (meth)acrylic acid monomer: and a poly(alkylene oxide) monomer optionally including a hydrocarbon chain. Embodiment 2 is the filtration media of embodiment 1 wherein the (meth)acrylic acid monomer is methacrylic acid.

Embodiment 3 is the filtration media of any of the previous embodiments wherein the poly(alkylene oxide) monomer has the formula:

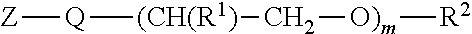

wherein: Z is a polymerizable ethylenically unsaturated group: Q is a divalent linking group (in certain embodiments, Q is selected from the group of —O—, —NR³—, —C(O)O—, and —C(O)NR³—, wherein R³ is H or CH₃ (preferably, R³ is H): in certain embodiments, Q is selected from the group of —O— and —NR³—, wherein R³ is H or CH₃ (preferably, R³ is H); and in certain embodiments, Q is —O—): R¹ is H or an alkyl group (preferably, a C1-C4 alkyl group): R² is H or an alkyl group (preferably, a C1-C40 alkyl group); and m is 2 to 100.

Embodiment 4 is the filtration media of embodiment 3 wherein Z is selected from the group consisting of:

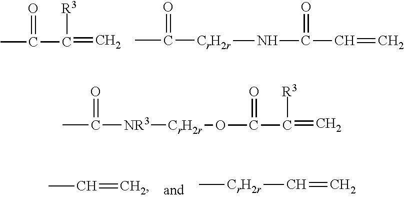

wherein: R³ is H or CH₃; and r is 1-10.

Embodiment 5 is the filtration media of any of the previous embodiments wherein the poly(alkylene oxide) monomer comprises a poly(alkylene oxide) (meth)acrylate monomer (wherein Z is —C(O)—C(R³)═CH₂).

Embodiment 6 is the filtration media of embodiment 4 or 5 wherein R³ is CH₃. Embodiment 7 is the filtration media of any of embodiments 3 through 6 wherein R¹ is H. Embodiment 8 is the filtration media of any of embodiments 3 through 7 wherein R² is a C1-C40 alkyl group (i.e., the poly(alkylene oxide) monomer includes a hydrocarbon chain of at least 1 carbons and up to 40 carbons). Embodiment 9 is the filtration media of any of embodiments 3 through 8 wherein m is 2 to 50.

Embodiment 10 is the filtration media of any of the previous embodiments wherein the poly(alkylene oxide) monomer is present in an amount of at least 2 wt-%, or at least 5 wt-%, and the (meth)acrylic acid monomer is present in an amount of at least 25 wt-%, or at least 50 wt-%, based on the total weight of the interpolymerized monomers. Embodiment 11 is the filtration media of any of the previous embodiments wherein the poly(alkylene oxide) monomer is present in an amount of up to 75 wt-%, or up to 50 wt-%, and the (meth)acrylic acid monomer is present in an amount of up to 98 wt-%, or up to 95 wt-%, based on the total weight of the interpolymerized monomers.

Embodiment 12 is the filtration media of any of the previous embodiments wherein the porous substrate is a porous polymeric substrate. Embodiment 13 is the filtration media of embodiment 12 wherein the porous polymeric substrate is a nonwoven substrate.

Embodiment 14 is an article comprising the viral filtration media of any of embodiments 1 through 13.

Embodiment 15 is a method of filtering a virus-containing sample, the method comprising: providing a viral filtration media comprising: a porous substrate comprising a surface having a polymer grafted thereto, wherein the grafted polymer comprises interpolymerized monomers comprising: a (meth)acrylic acid monomer: and, optionally, a poly(alkylene oxide) monomer (optionally including a hydrocarbon chain): providing a virus-containing sample comprising a target virus: and contacting the viral filtration media with the virus-containing sample under conditions effective to separate at least a portion of the target virus from other material in the virus-containing sample.

Embodiment 16 is the method of embodiment 15 wherein the viral filtration media is incorporated in a filter element. Embodiment 17 is the method of embodiment 15 or 16 wherein contacting comprises allowing a moving virus-containing sample to impinge upon an upstream surface of the filter element (or the filtration media) for a time sufficient to effect separation of at least a portion of the target virus from other material in the virus-containing sample. Embodiment 18 is the method of embodiment 17 wherein contacting comprises allowing a moving virus-containing sample to impinge upon an upstream surface of the filter element (or the filtration media) for a time sufficient to effect binding of at least a portion of the target virus in the virus-containing sample to the filtration media. Embodiment 19 is the method of embodiment 18 wherein contacting comprises allowing a moving virus-containing sample to impinge upon an upstream surface of the filter element (of the filtration media) for a time sufficient to effect binding of at least a portion of other material in the virus-containing sample to the filtration media. Embodiment 20 is the method of any one of embodiments 17 through 19 wherein contacting comprises allowing a moving virus-containing sample to impinge upon an upstream surface of the filter element (of the filtration media) for a time sufficient to effect separation of at least a portion of the target virus from another virus in the virus-containing sample.

Embodiment 21 is the method of any of embodiments 15 through 20 wherein the (meth)acrylic acid monomer is methacrylic acid.

Embodiment 22 is the method of any of embodiments 15 through 21 wherein the poly(alkylene oxide) monomer has the formula:

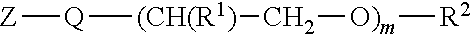

wherein: Z is a polymerizable ethylenically unsaturated group: Q is a divalent linking group (in certain embodiments, Q is selected from the group of —O—, —NR³—, —C(O)O—, and —C(O)NR³—, wherein R³ is H or CH₃ (preferably, R³ is H): in certain embodiments, Q is selected from the group of —O— and —NR³—, wherein R³ is H or CH₃ (preferably, R³ is H); and in certain embodiments, Q is —O—): R¹ is H or an alkyl group (preferably, a C1-C4 alkyl group): R² is H or an alkyl group (preferably, a C1-C40 alkyl group): and m is 2 to 100.

Embodiment 23 is the method of embodiment 22 wherein Z is selected from the group consisting of:

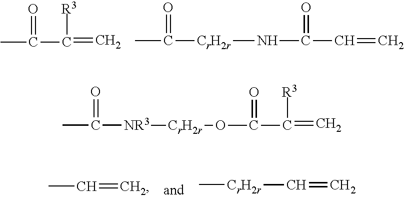

wherein: R³ is H or CH₃: and r is 1-10.

Embodiment 24 is the method of embodiments 22 or 23 wherein the poly(alkylene oxide) monomer comprises a poly(alkylene oxide) (meth)acrylate monomer (wherein Z is —C(O)—C(R³)═CH₂).

Embodiment 25 is the method of embodiment 23 or 24 wherein R³ is CH₃. Embodiment 26 is the method of any of embodiments 22 through 25 wherein R¹ is H. Embodiment 27 is the method of any of embodiments 22 through 26 wherein R² is an alkyl group (preferably, a C1-C40 alkyl group). Embodiment 28 is the method of any of embodiments 22 through 27 wherein m is 2 to 50.

Embodiment 29 is the method of any of embodiments 22 through 28 wherein the poly(alkylene oxide) monomer is present in an amount of at least 2 wt-%, or at least 5 wt-%, and the (meth)acrylic acid monomer is present in an amount of at least 25 wt-%, or at least 50 wt-%, based on the total weight of the interpolymerized monomers. Embodiment 30 is the method of any of embodiments 22 through 29 wherein the poly(alkylene oxide) monomer is present in an amount of up to 75 wt-%, or up to 50 wt-%, and the (meth)acrylic acid monomer is present in an amount of up to 98 wt-%, or up to 95 wt-%, based on the total weight of the interpolymerized monomers.

Embodiment 31 is the method of any of embodiments 15 through 30 wherein the porous substrate is a porous polymeric substrate. Embodiment 32 is the method of embodiment 31 wherein the porous polymeric substrate is a nonwoven substrate. Embodiment 33 is the method of any of embodiment 15 through 32 wherein the virus-containing sample is a biopharmaceutical drug, protein product or viral vector product, cell culture fluid containing a biopharmaceutical drug, protein product, or a viral vector product, clarified cell culture fluid containing a biopharmaceutical drug, protein product, or a viral vector product.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

Unless otherwise noted, all parts, percentages, ratios, etc. in the examples and the rest of the specification are by weight, and all reagents used in the examples were obtained, or are available, from general chemical suppliers, or may be synthesized by conventional methods.

### Materials and Methods

### Preparation of Phi-X174 Virus Stock Culture

Phi-X174 bacteriophage (ATCC 13706-B1) was obtained from ATCC (Manassas, VA). The virus culture was produced by growing a one liter culture of E. coli (ATCC 13706) in CRITERION Nutrient Broth (product No. C6471, Hardy Diagnostics, Santa Maria, CA) plus 5% sodium chloride at 37° C. with shaking to an OD of 0.45. The culture was inoculated with 1010 plaque forming units (pfu) of Phi-X174 virus. The inoculated culture was grown for an additional 4 hours at 37° C. with shaking at 210 revolutions per minute (rpm). Cells were removed by centrifugation at 3700×g and the supernatant was filtered through a 0.2 micron polyethersulfone (PES) bottle top membrane filter (FISHERBRAND product No. FB12566506 obtained from Thermo Fisher Scientific, Waltham, MA).

### Preparation of Phi 6 Virus Stock Culture

Phi 6 bacteriophage (DSM 21518) was obtained from the DSMZ German Collection of Microorganisms and Cell Cultures, Braunschweig, Germany. The virus culture was produced by inoculating 100 mL of tryptic soy broth (Hardy Diagnostics) plus 5 mM magnesium sulfate with 1.5 mL of Pseudomonas syringae (DSM 21482, obtained from the DSMZ German Collection of Microorganisms and Cell Cultures) overnight culture. The culture was grown at 25° C. with shaking at 210 rpm for 2 hours. The culture was then inoculated with 109 plaque forming units (pfu) of Phi 6 virus. The inoculated culture was grown for an additional 3 hours at 25° C. with shaking at 210 rpm. Cells were removed by centrifugation at 3700×g and the supernatant was filtered through a 0.2 micron PES bottle top membrane filter.

### Preparation of PR772 Virus Stock Culture

PR772 virus (ATCC BAA-769-B1) was diluted to 106 pfu/mL. Molten tryptic soy top agar (0.9% agar, 2.5 mL) was mixed with 100 microliters of diluted virus and 100 microliters of E. coli BAA769 host bacteria (ATCC BAA-769) overnight culture. The mixture was poured on top of a 100×15 mm tryptic soy agar petri plate and incubated overnight at 37° C. The procedure was repeated to prepare a total of 10 plates. After incubation, 2.5 mL of 1X phosphate buffered saline pH 7.4 (PBS) was added to each plate, and the top agar was scraped into a 50 ml conical tube. The tube was incubated for 4 hours at 20-25° C. with gentle agitation on an orbital shaker. The tube was then centrifuged at 3700×g for 20 minutes and the supernatant was filtered through a 0.2 micron PES membrane filter.

Preparation of Lentivirus Culture Lentivirus was produced in HEK-293 suspension cells using a Gibco LV-MAX Lentiviral Production System according to the manufacturer's instructions (Thermo Fisher Scientific). The lentivirus culture had a cell count of 6 million cells/mL at 50% viability and a virus titer of 1.54×10⁸ infectious units per mL (IFU/mL). Virus titer was determined using a QUICKTITER Lentivirus Titer kit (VPK-107) obtained from Cell Biolabs (San Diego, CA) according to the manufacturer's instructions. The turbidity of the culture was 268 nephelometric turbidity units (NTU), measured with an Orion AQ4500 turbidity meter (Thermo Fisher Scientific).

### Determination of Phi-X174 Virus Concentration by Plaque Assay

Phi-X174 virus samples were serially diluted (10-fold) with 1×PBS buffer (pH 7.4). Molten top agar (nutrient broth with 0.9% agar, 2.5 mL) was mixed with 50 microliters of E. coli (ATCC 13706) host bacteria overnight culture and 100 microliters of diluted Phi-X174 virus. The mixture was poured on top of a 100 mm nutrient agar petri plate and incubated for 3-4 hours at 37° C. Following incubation, the plaque-forming units (pfu) were counted. The number of pfu was correlated with virus particle number. The virus particle concentration (particles/mL) was calculated from the PFU count adjusted for dilution.

### Determination of PR772 Virus Concentration by Plaque Assay

PR772 virus samples were serially diluted (10-fold) with 1×PBS buffer (pH 7.4). Molten tryptic soy top agar (tryptic soy broth with 0.9% agar, 2.5 mL) was mixed with 50 microliters of E. coli BAA769 host bacteria overnight cell culture and 100 microliters of diluted PR772 virus. The mixture was poured on top of a standard tryptic soy agar plate and incubated overnight at 37° C. Following incubation, the plaque-forming units (pfu) were counted. The number of pfu was correlated with virus particle number. The virus particle concentration (particles/mL) was calculated from the pfu count adjusted for dilution.

### Determination of Phi 6 Virus Concentration by Plaque Assay

Phi 6 virus samples were serially diluted (10-fold) with 1×PBS buffer (pH 7.4). Molten tryptic soy top agar (2.5 mL of tryptic soy broth with 5 mM MgSO₄ and 0.9% agar) was mixed with 50 microliters of Pseudomonas syringae host bacteria overnight cell culture and 100 microliters of diluted Phi 6 virus. The mixture was poured on top of a standard tryptic soy agar plate and incubated overnight at 25° ° C. Following incubation, the plaque-forming units (pfu) were counted. The number of pfu was correlated with virus particle number. The virus particle concentration (particles/mL) was calculated from the PFU count adjusted for dilution.

### Preparation of Chinese Hamster Ovary (CHO) Cell Culture

Chinese hamster ovary (CHO) cells producing an IgG monoclonal antibody were cultured in suspension from frozen cell stock to a series of flask seeding cultures in a CO₂ incubator, followed by a fed-batch culture process using a Wave bioreactor (GE Healthcare, Chicago, IL) and a 50 L disposable cell bag (with pH control and dissolved oxygen monitoring). HYCLONE PF-CHO LS cell culture media (obtained from Cytiva, Marlborough, MA) was used. CHO cell cultures were harvested during stationary phase, typically on day 12. The cell culture was clarified by filtering sequentially through a 3M ZETA PLUS 05SP01 depth filter, a 3M ZETA PLUS 10SP02A depth filter (obtained from the 3M Company, Maplewood, MN), and then a 0.2-micron PES bottle top filter (obtained from Thermo Fisher Scientific).

### Monomers for Grafting Solutions

Methacrylic acid (MAA, CAS No. 79-41-4) was obtained from the MilliporeSigma Company, St. Louis, MO.

Poly(ethylene glycol) behenyl ether methacrylate (PEGBEMA) solution (CAS No. 125441-87-4, product No. 468258, Mn about 1500 g/mol) was obtained from the MilliporeSigma Company as a solution containing 50 wt-% PEGBEMA, 25 wt-% MAA, and 25 wt-% water.

Poly(ethylene glycol) methyl ether methacrylate having a number-average molecular weight of 950 g/mol (PEGMA-950, CAS No. 26915-72-0, product No. 447951) was obtained from the MilliporeSigma Company.

Poly(ethylene glycol) methyl ether methacrylate having a number-average molecular weight of 300 g/mol (PEGMA-300, CAS No. 26915-72-0, product No. 447935) was obtained from the MilliporeSigma Company.

Poly(ethylene glycol) methacrylate having a number-average molecular weight of 360 g/mol (PEGMA-360, CAS No. 25736-86-1, product No. 409537) was obtained from the MilliporeSigma Company.

Poly(ethylene glycol) methyl ether methacrylate having a number-average molecular weight of 2000 g/mol (PEGMA-2000, CAS No. 26915-72-0, product No. 457876) was obtained from the MilliporeSigma Company.

### Grafting Solutions

Individual aqueous grafting solutions were prepared with the monomer compositions reported in Tables 1 and 1A. All components in the grafting solutions are reported in weight percent (wt-%).

TABLE 1

| Grafting | Concentration of Component in Grafting Solution (wt-%) | | | | |
|---|---|---|---|---|---|
| Solution | MAA | PEGBEMA | PEGMA-950 | PEGMA-300 | Water |
| A | 26.3 | 0 | 0 | 0 | 73.7 |
| B | 23.7 | 2.6 | 0 | 0 | 73.7 |
| C | 19.7 | 6.6 | 0 | 0 | 73.7 |
| D | 13.2 | 13.2 | 0 | 0 | 73.6 |
| E | 8.8 | 17.5 | 0 | 0 | 73.7 |
| F | 11.6 | 0 | 14.7 | 0 | 73.7 |
| G | 22.2 | 0 | 0 | 0 | 77.8 |
| H | 7.4 | 14.8 | 0 | 0 | 77.8 |
| I | 9.8 | 0 | 12.4 | 0 | 77.8 |
| J | 15.9 | 0 | 0 | 6.4 | 77.7 |

TABLE 1A

| Grafting | Molar Ratio of MAA to |
|---|---|
| Solution | Poly(alkylene oxide) Monomer |
| B | 159:1 |
| C | 52:1 |
| D | 17.4:1 |
| E | 8.8:1 |
| F | 8.7:1 |
| H | 8.7:1 |
| I | 8.7:1 |
| J | 8.7:1 |

Individual aqueous grafting solutions were prepared with the monomer compositions reported in Table 1B and 1C. All components in the grafting solutions are reported in weight percent (wt-%).

TABLE 1B

| | | Concentration of Component in | | | |
|---|---|---|---|---|---|
| | | Grafting Solution (wt. %) | | | |
| Grafting | | PEGMA- | PEGMA- | PEGMA- | |
| Solution | MAA | 2000 | 950 | 360 | Water |
| K | 20 | 0 | 0 | 5 | 75 |
| L | 20 | 0 | 0 | 15 | 65 |
| M | 20 | 0 | 0 | 25 | 55 |
| N | 20 | 0 | 5 | 0 | 75 |
| O | 20 | 0 | 15 | 0 | 65 |
| P | 20 | 0 | 25 | 0 | 55 |
| Q | 20 | 5 | 0 | 0 | 75 |
| R | 20 | 15 | 0 | 0 | 65 |
| S | 20 | 25 | 0 | 0 | 55 |

TABLE 1C

| Grafting | Molar Ratio of MAA to |
|---|---|
| Solution | Poly(alkylene oxide) Monomer |
| K | 16.7:1 |
| L | 5.6:1 |
| M | 3.3:1 |
| N | 44.2:1 |
| O | 14.7:1 |
| P | 8.8:1 |
| Q | 93:1 |
| R | 31:1 |
| S | 18.6:1 |

### Example 1. Preparation of Functionalized Nonwoven A (FNW-A)

A melt-blown polypropylene nonwoven web (having an effective fiber diameter of 4.6 micrometers, basis weight of 262.8 grams per square meter (gsm), solidity of 8.8%) was grafted with nitrogen purged Grafting Solution A.

A sample of the nonwoven web (17.8 cm by 22.9 cm) was placed in a glove box and purged of air under a nitrogen atmosphere. Once the oxygen levels reached less than 20 ppm, the nonwoven substrate was inserted into a plastic bag and the bag was sealed.

Grafting solution A (150 grams) was added to a glass jar. The jar was capped and shaken by hand to mix the contents. The jar was then opened and the solution was sparged with nitrogen for 2 minutes to remove any dissolved oxygen from the solution. The jar was re-capped and transferred into the oxygen depleted glovebox. The jar lid was then removed to flush any residual air from the jar headspace.

The sealed bag containing nonwoven sample was removed from the glove box and irradiated with an electron beam (Electrocure, Energy Sciences Inc, Wilmington, MA) at an accelerating voltage of 300 kV to a dose of 10 Mrad. The bag containing the irradiated nonwoven sample was then returned to the glove box and purged of air as described above.

Grafting Solution A (100 g) was added to the plastic bag containing the nonwoven sample. The bag was sealed and the solution was distributed through the nonwoven sample using a hand roller so that the nonwoven sheet was uniformly covered with the solution. The nonwoven sample was maintained flat in the sealed bag for 3 hours and then the bag was removed from the glove box. The resulting polymer-grafted nonwoven sample was removed from the bag and boiled in deionized water for one hour. The sample was removed from the water bath and air dried at room temperature for 24 hours. The dried polymer-grafted nonwoven sample was labeled as Functionalized Nonwoven A (FNW-A). Discs (27 mm in diameter) were punched from the dried sample.

The amount (g) of monomer grafted to the nonwoven sample as grafted polymer was measured gravimetrically by weighing the nonwoven sample before and after the grafting procedure. The weight after the grafting procedure was measured following the final drying step and before discs were punched from the sample. The weight of grafted polymer was then compared to the total weight of the monomers in the grafting solution to determine the percentage of monomer in the grafting solution that was incorporated in the grafted polymer of the functionalized nonwoven. For FNW-A, 96% of the monomers were incorporated in the grafted polymer.

### Example 2. Preparation of Functionalized Nonwoven B (FNW-B)

The same procedure as described in Example 1 was followed with the exception that Grafting Solution B was used, instead of Grafting Solution A. For FNW-B, 97% of the monomers were incorporated in the grafted polymer.

### Example 3. Preparation of Functionalized Nonwoven C (FNW-C)

The same procedure as described in Example 1 was followed with the exception that Grafting Solution C was used, instead of Grafting Solution A. For FNW-C, 93% of the monomers were incorporated in the grafted polymer.

### Example 4. Preparation of Functionalized Nonwoven D (FNW-D)

The same procedure as described in Example 1 was followed with the exception that Grafting Solution D was used, instead of Grafting Solution A. For FNW-D, 90% of the monomers were incorporated in the grafted polymer.

### Example 5. Preparation of Functionalized Nonwoven E (FNW-E)

The same procedure as described in Example 1 was followed with the exception that Grafting Solution E was used, instead of Grafting Solution A. For FNW-E, 93% of the monomers were incorporated in the grafted polymer.

### Example 6. Preparation of Functionalized Nonwoven F (FNW-F)

The same procedure as described in Example 1 was followed with the exception that Grafting Solution F was used, instead of Grafting Solution A. For FNW-F, 87% of the monomers were incorporated in the grafted polymer.

### Example 7. Preparation of Functionalized Nonwoven G (FNW-G)

A melt-blown polypropylene nonwoven web (having an effective fiber diameter of 8 micrometers, basis weight of 200 grams per square meter (gsm), solidity of 10%) was grafted with nitrogen purged Grafting Solution G.

A sample of the nonwoven web (17.8 cm by 22.9 cm) was placed in a glove box and purged of air under a nitrogen atmosphere. Once the oxygen levels reached less than 20 ppm, the nonwoven substrate was inserted into a plastic bag and the bag was sealed.

Grafting solution G (150 grams) was added to a glass jar. The jar was capped and shaken by hand to mix the contents. The jar was then opened and the solution was sparged with nitrogen for 2 minutes to remove any dissolved oxygen from the solution. The jar was re-capped and transferred into the oxygen depleted glovebox. The jar lid was then removed to flush any residual air from the jar headspace.

The sealed bag containing nonwoven sample was removed from the glove box and irradiated with an electron beam (Electrocure, Energy Sciences Inc, Wilmington, MA) at an accelerating voltage of 300 kV to a dose of 10 Mrad. The bag containing the irradiated nonwoven sample was then returned to the glove box and purged of air as described above.

Grafting solution G (90 g) was added to the plastic bag containing the nonwoven sample. The bag was sealed and the solution was distributed through the nonwoven sample using a hand roller so that the nonwoven sheet was uniformly covered with the solution. The nonwoven sample was maintained flat in the sealed bag for 3 hours and then the bag was removed from the glove box. The resulting polymer-grafted nonwoven sample was removed from the bag and boiled in deionized water for one hour. The sample was removed from the water bath and air dried at room temperature for 24 hours. The polymer-grafted nonwoven sample was labeled as Functionalized Nonwoven G (FNW-G). Discs (27 mm in diameter) were punched from the dried sample. The percentage of monomer in the grafting solution that was incorporated in the grafted polymer of the functionalized nonwoven was determined gravimetrically as described in Example 1. For FNW-G, all of the monomers were incorporated in the grafted polymer.

### Example 8. Preparation of Functionalized Nonwoven H (FNW-H)

The same procedure as described in Example 7 was followed with the exception that Grafting Solution H was used, instead of Grafting Solution G. For FNW-H, 88% of the monomers were incorporated in the grafted polymer.

### Example 9. Preparation of Functionalized Nonwoven I (FNW-I)

The same procedure as described in Example 7 was followed with the exception that Grafting Solution I was used, instead of Grafting Solution G. For FNW-I, 91% of the monomers were incorporated in the grafted polymer.

### Example 10. Preparation of Functionalized Nonwoven J (FNW-J)

The same procedure as described in Example 7 was followed with the exception that Grafting Solution J was used, instead of Grafting Solution G. For FNW-J, all of the monomers were incorporated in the grafted polymer.

### Example 11

A plastic filtration capsule was used. The capsule consisted of a sealed, circular housing. The capsule housing was prepared from two halves (upper and lower halves) which were mated and sealed together at the perimeter after the filtration elements were inserted in the internal cavity of the lower housing. Fluid inlet and vent ports were located on the upper portion of the housing and a fluid outlet port was located on the lower portion of the housing. The outlet port was centered in the middle of the lower housing surface. Two discs (27 mm diameter) of TYPAR 3161L polypropylene spunbond nonwoven (10 mil thick, obtained from Fiberweb, Inc., Old Hickory, TN) were placed in the bottom of the lower housing. A single disc (27 mm diameter) of a MICRO-PES Flat Type 2F polyethersulfone membrane with a 0.2 micrometer nominal pore size (obtained from the 3M Company) was placed on top of the nonwoven layer. The nonwoven and membrane layers were ultrasonically welded at the margins to the bottom inner surface of the lower housing. Two discs (27 mm diameter) selected from the Functionalized Nonwovens A-F (prepared above) were then placed on top of the membrane disc. A polypropylene spacer ring (25.4 mm OD, 21.84 mm ID, 50 mil thick) was inserted between the functionalized nonwoven discs. In all constructions of filtration capsules, the two functionalized nonwoven discs included in a capsule were the same type (i.e. were prepared with the same grafting solution). The upper and lower housings were mated together and ultrasonically welded using a Branson 20 kHz Ultrasonic welder (Model 2000xdt, Emerson Electric Company, St. Louis, MO) to form a finished filter capsule.

The overall outer diameter of the finished capsule was about 4.3 cm and the overall height including inlet, outlet, and vent ports was about 5.9 cm. The effective filtration area of the capsule was 4.0 cm² and the bed volume of the nonwoven media was 1.4 mL.

The capsule was flushed with 20 mL of 1×PBS buffer (pH 7.4) at 1 mL/minute using a Masterflex L/S peristaltic pump (Cole-Parmer, Vernon Hills, IL).

The virus challenge solution contained 1.2×10⁸ pfu/mL of Phi 6 virus, 1.9×10⁸ pfu/mL Phi-X174, and 1.7×10⁸ pfu/mL of PR 772 virus in CHO clarified cell culture fluid. The challenge solution was prepared by spiking the CHO culture described above with samples from the virus stock cultures (described above).

Any residual PBS buffer from the capsule flushing step was removed from the headspace of the capsule and 20 mL of the virus challenge solution was pumped through the capsule at 1 mL/minute. The resulting filtrate was collected. Virus concentrations of the input challenge solution and the filtrate sample were measured for each virus using the corresponding plaque assays described above. The log reduction values (LRV) from filtration were calculated by comparing the virus concentrations of the filtrate samples to the virus concentrations in the input challenge solutions before filtration (Equation A). The results are reported in Table 2 from a single trial for each capsule.

$$LRV = log 10 [ \frac{concentration of virus in sample before filtration ( pfu / m l )}{concentration of virus in the filtrate ( pfu / ml )} ] \qquad Equation A$$

All of the capsules tested had significantly greater log reduction values for the enveloped Phi 6 virus than for the nonenveloped PR772 and Phi-X174 viruses.

TABLE 2

| | | Log Reduction Values for | | |
|---|---|---|---|---|
| Functionalized | | Viruses in Challenge | | |
| Nonwoven | Monomer Composition of Grafting | Solution using Filtration | | |
| (FNW) used | Solution used to prepare FNW | Capsule (Example 11) | | |
| in Capsule | (weight %) | Phi 6 | PR772 | Phi-X174 |
| FNW-A | MAA (26.3%) | >7.1* | 2.5 | 1.6 |
| FNW-B | MAA (23.7%), PEGBEMA (2.6%) | >7.1* | 0.5 | 1.6 |
| FNW-C | MAA (19.7%), PEGBEMA (6.6%) | >7.1* | 1.4 | 1.6 |
| FNW-D | MAA (13.2%), PEGBEMA (13.2%) | 3.7 | 0 | 0.38 |
| FNW-E | MAA (8.8%), PEGBEMA (17.5%) | >7.1* | 0 | 0.16 |
| FNW-F | MAA (11.6%), PEGMA-950 (14.7%) | 3 | 0 | 0.16 |

*no pfu observed in the undiluted filtrate sample

### Example 12

The same procedure a reported in Example 11 was followed with the exception that different Functionalized Nonwovens were used in the filtration capsules and a different virus challenge solution was used. Capsules were prepared as in Example 11 using two discs (27 mm diameter) selected from the Functionalized Nonwovens G-J (prepared above). In all constructions of filtration capsules, the two functionalized nonwoven discs included in a capsule were the same type (i.e. were prepared with the same grafting solution). The virus challenge solution contained 1.2×10⁷ pfu/mL of Phi 6 virus and 1.6×10⁸ pfu/mL of PR772 virus in CHO clarified cell culture fluid. The log reduction values (LRV) from filtration were calculated by comparing the virus concentrations of the filtrate samples to the virus concentrations in the input challenge solutions before filtration (Equation A). The LRV results are reported in Table 3 as the mean value of two replicates (n=2) for each capsule.

TABLE 3

| | | Log Reduction Values for | |
|---|---|---|---|
| Functionalized | | Viruses in Challenge | |
| Nonwoven | Monomer Composition of Grafting | Solution using Filtration | |
| (FNW) used | Solution used to prepare FNW | Capsule (Example 12) | |
| in Capsule | (weight %) | Phi 6 | PR772 |
| FNW-G | MAA (22.2%) | 4.0 | 3.7 |
| FNW-H | MAA (7.4%), PEGBEMA (14.8%) | 2.4 | 0 |
| FNW-I | MAA (9.8%), PEGMA-950 (12.4%) | 7.0 | 0.6 |
| FNW-J | MAA (15.9%), PEGMA-300 (6.4%) | 7.0 | 5.3 |

### Example 13

Individual filtration capsules were prepared as in Example 11 with the exception that a single disc (27 mm diameter) of DURAPES 600 polyethersulfone membrane with a 0.6 micrometer nominal pore size (obtained from the 3M Company) was used in place of the MICRO-PES Flat Type 2F polyethersulfone membrane and different Functionalized Nonwovens were used. Two discs (27 mm diameter) were selected from the Functionalized Nonwovens G-J (prepared above). In all constructions of filtration capsules, the two functionalized nonwoven discs included in a capsule were the same type (i.e. were prepared with the same grafting solution). The Lentivirus cell culture described above was used as the feed culture.

Each capsule was flushed with 20 mL of 1×PBS buffer (pH 7.4) at 1 mL/minute using a Masterflex L/S peristaltic pump (Cole-Parmer, Vernon Hills, IL). Any residual PBS buffer from the capsule flushing step was removed from the headspace of the capsule. The lentivirus culture was pumped through the capsule at 0.5 mL/minute and the resulting filtrate was collected until a differential pressure of 15 psi was reached. The throughput was calculated using the volume collected. The throughput values (L/m²) for lentivirus culture filtered using different filtration capsules (i.e. capsules with different media) are reported in Table 4.

The turbidity of the culture before and after filtration with the filtration capsule was measured using an Orion AQ4500 turbidity meter (Thermo Fisher Scientific) in Nephelometric Turbidity Units (NTU). The turbidity of the lentivirus culture before filtration through the filtration capsule was 268 NTU. The results for reduction in turbidity of lentivirus cultures after filtration through a capsule are reported in Table 5.

Following the turbidity measurement, the lentivirus titer of the filtrate from each filtration capsule was determined by first filtering each filtrate sample through a 4.9 cm^{2 0.2} micron PES membrane syringe filter (Pall Corporation, Port Washington, NY) and then analyzing the resulting filtrate from the syringe filter using a LENTI-X p24 Rapid Titer Assay kit (Takara Bio, Mountain View, CA).

The percentage of lentivirus recovered from the filtration procedure was determined by comparing the lentivirus titer of the filtrate sample to the lentivirus titer of the starting culture (i.e. the feed culture). Prior to analysis, the feed culture was filtered through a 0.45 micron PES membrane filter (FISHERBRAND, obtained from Thermo Fisher Scientific) using vacuum filtration. The results for percent recovery of lentivirus after filtration of lentivirus culture using different filtration capsules are reported in Table 6.

In Tables 4-6, the results for capsules containing FNW-G are reported as the mean value of three replicates (n=3) and the results for capsules containing FNW—H, FNW-I, FNW-J are reported from a single trial for each capsule.

TABLE 4

| Functionalized | Monomer Composition | Lentivirus Culture |
|---|---|---|
| Nonwoven | of Grafting Solution | Throughput using the |
| (FNW) used | used to prepare FNW | Filtration Procedure |
| in Capsule | (weight %) | (L/m²) |
| FNW-G | MAA (22.2%) | 79.7 |
| FNW-H | MAA (7.4%), | 63.7 |
| | PEGBEMA (14.8%) | |
| FNW-I | MAA (9.8%), | 56.3 |
| | PEGMA-950 (12.4%) | |
| FNW-J | MAA (15.9%), | 19.0 |
| | PEGMA-300 (6.4%) | |

TABLE 5

| Functionalized | Percent Reduction in Turbidity |
|---|---|
| Nonwove (nFNW) | of Lentivirus Culture Sample |
| used in Capsule | using the Filtration Procedure |
| FNW-G | 96% |
| FNW-H | 98% |
| FNW-I | 97% |
| FNW-J | 98% |

TABLE 6

| Functionalized | Percent Lentivirus Recovered |
|---|---|
| Nonwoven (FNW) | from Culture using the |
| used in Capsule | Filtration Procedure |
| FNW-G | 0% |
| FNW-H | 69% |
| FNW-I | 81% |
| FNW-J | 18% |

### Example 14. Preparation of Functionalized Nonwovens K to S (FNW-K to FNW-S)

A melt-blown polypropylene nonwoven web (having an effective fiber diameter of 12 micrometers, basis weight of 200 grams per square meter (gsm), solidity of 10%) was grafted with a single nitrogen purged grafting solution selected from one of the nine Grafting Solutions K to S described in Table 1B.

A sample of the nonwoven web (17.8 cm by 22.9 cm) was placed in a glove box and purged of air under a nitrogen atmosphere. Once the oxygen levels reached less than 20 ppm, the nonwoven substrate was inserted into a plastic bag and the bag was sealed.

The selected grafting solution (150 grams) was added to a glass jar. The jar was capped and shaken by hand to mix the contents. The jar was then opened and the solution was sparged with nitrogen for 2 minutes to remove any dissolved oxygen from the solution. The jar was re-capped and transferred into the oxygen depleted glovebox. The jar lid was then removed to flush any residual air from the jar headspace.

The sealed bag containing nonwoven sample was removed from the glove box and irradiated with an electron beam (Electrocure, Energy Sciences Inc.) at an accelerating voltage of 300 kV to a dose of 10 Mrad. The bag containing the irradiated nonwoven sample was then returned to the glove box and purged of air as described above.

The selected grafting solution (90 g) was added to the plastic bag containing the nonwoven sample. The bag was sealed and the solution was distributed through the nonwoven sample using a hand roller so that the nonwoven sheet was uniformly covered with the solution. The nonwoven sample was maintained flat in the sealed bag for 3 hours. The resulting polymer-grafted nonwoven sample was removed from the bag and boiled in deionized water for one hour. The sample was removed from the water bath and air dried at room temperature for 24 hours. The polymer-grafted nonwoven article was labeled with the same letter designation as the corresponding grafting solution used. For example, Functionalized Nonwoven K (FNW-K) was prepared using Grafting Solution K and Functionalized Nonwoven N (FNW-N) was prepared using Grafting Solution N. Discs (27 mm in diameter) were punched from the dried sample. The percentage of monomer in the grafting solution that was incorporated in the grafted polymer of each of the functionalized nonwoven was determined gravimetrically as described in Example 1 and the results are reported in Table 7.

TABLE 7

| Functionalized | Percentage of Monomers from |
|---|---|
| Nonwoven | Grafting Solution Incorporated |
| (FNW) | in the Grafted Polymer |
| FNW-K | 100% |
| FNW-L | 96% |
| FNW-M | 79% |
| FNW-N | 100% |
| FNW-O | 100% |
| FNW-P | 88% |
| FNW-Q | 100% |
| FNW-R | 68% |
| FNW-S | 61% |

### Example 15

The same procedure for preparing filtration capsules as reported in Example 11 was followed with the exception that different Functionalized Nonwovens were used in the filtration capsules. Capsules were prepared as in Example 11 using two discs (27 mm diameter) selected from the Functionalized Nonwovens K-M (prepared above). In all constructions of filtration capsules, the two functionalized nonwoven discs included in a capsule were the same type (i.e. were prepared with the same grafting solution). Each finished capsule was flushed with 20 mL of 1X pH 7.4 PBS buffer at 1 mL/min using a Masterflex L/S peristaltic pump (Cole-Parmer).

The virus challenge solution contained 1.1×10⁸ pfu/mL of Phi 6 virus and 9×10⁶ pfu/mL of PR 772 virus in 1×PBS buffer. The challenge solution was prepared by spiking the 1X PBS buffer with samples from the virus stock cultures (described above). The log reduction values (LRV) from filtration were calculated by comparing the virus concentrations of the filtrate samples to the virus concentrations in the input challenge solutions before filtration (Equation A in Example 11). Two capsules were prepared and tested for each functionalized nonwoven sample except for FWN-M in which only one capsule was tested. The results are reported in Table 8.

TABLE 8

| | Log Reduction Values (LRV) for Two Separate | | | |
|---|---|---|---|---|
| | Viruses in the Challenge Solution | | | |
| Functionalized | using a Filtration Capsule (Example 15) | | | |
| Nonwoven used | Capsule 1 | Capsule 2 | Capsule 1 | Capsule 2 |
| in Capsule | Phi 6 LRV | Phi 6 LRV | PR772 LRV | PR772 LRV |
| FNW-K | 7.0 | 7.0 | 4.1 | 4.1 |
| FNW-L | 7.0 | 7.0 | 0.4 | 0.4 |
| FNW-M | 7.0 | NT | 0.4 | NT |

NT = Not Tested

### Example 16

The same procedure a reported in Example 15 was followed with the exception that different Functionalized Nonwovens were used in the filtration capsules. Capsules were prepared as in Example 15 using two discs (27 mm diameter) selected from the Functionalized Nonwovens N-P (prepared above). The capsules were flushed with 20 mL of 1X pH 7.4 PBS buffer at 1 mL/min using a Masterflex L/S peristaltic pump (Cole-Parmer). The virus challenge solution contained 2.3×10⁷ pfu/mL of Phi 6 virus and 1.1×10⁶ pfu/mL of PR772 virus in 1×PBS buffer. The challenge solution was prepared by spiking the 1X PBS buffer with samples from the virus stock cultures (described above). The log reduction values (LRV) from filtration were calculated by comparing the virus concentrations of the filtrate samples to the virus concentrations in the input challenge solutions before filtration (Equation A in Example 11). Two capsules were prepared and tested for each functionalized nonwoven sample except for FWN-P in which only one capsule was tested. The results are reported in Table 9.

TABLE 9

| | Log Reduction Values (LRV) for Two Separate | | | |
|---|---|---|---|---|
| | Viruses in the Challenge Solution | | | |
| Functionalized | using a Filtration Capsule (Example 16) | | | |
| Nonwoven used | Capsule 1 | Capsule 2 | Capsule 1 | Capsule 2 |
| in Capsule | Phi 6 LRV | Phi 6 LRV | PR772 LRV | PR772 LRV |
| FNW-N | 6.4 | 6.4 | 6.0 | 4.08 |
| FNW-O | 6.4 | 6.4 | 0.3 | 0.1 |
| FNW-P | 6.4 | NT | 0.6 | NT |

NT = Not Tested

### Example 17

The same procedure a reported in Example 15 was followed with the exception that different Functionalized Nonwovens were used in the filtration capsules. Capsules were prepared as in Example 15 using two discs (27 mm diameter) selected from the Functionalized Nonwovens Q-S (prepared above). The capsules were flushed with 20 mL of 1X pH 7.4 PBS buffer at 1 mL/min using a Masterflex L/S peristaltic pump (Cole-Parmer). The virus challenge solution for samples contained 1.3×10⁷ pfu/mL of Phi 6 virus and 1.3×10⁷ pfu/mL of PR772 virus in 1×PBS buffer. The challenge solution was prepared by spiking the 1X PBS buffer with samples from the virus stock cultures (described above). The log reduction values (LRV) from filtration were calculated by comparing the virus concentrations of the filtrate samples to the virus concentrations in the input challenge solutions before filtration (Equation A in Example 11). Two capsules were prepared and tested for each functionalized nonwoven sample except for of FWN-S in which only one capsule was tested. The results are reported in Table 10.

TABLE 10

| | Log Reduction Values for Two Separate | | | |
|---|---|---|---|---|
| | Viruses in the Challenge Solution | | | |
| Functionalized | using a Filtration Capsule (Example 17) | | | |
| Nonwoven used | Capsule 1 | Capsule 2 | Capsule 1 | Capsule 2 |
| in Capsule | Phi 6 LRV | Phi 6 LRV | PR772 LRV | PR772 LRV |
| FNW-Q | 6.1 | 6.1 | 3.5 | 3.5 |
| FNW-R | 6.1 | 6.1 | 0 | 0.3 |
| FNW-S | 6.1 | NT | 0 | NT |

NT = Not Tested

### Example 18

The reactive component of all monomers used for polymer grafting was a methacrylate group. For the preparation of FNWs A-S, the PAO monomer used was a poly(ethylene glycol) methyl ether methacrylate or a poly(ethylene glycol) methacrylate represented by the abbreviation of PEGMA (i.e., selected from the monomers designated PEGMA-300, PEGMA-360, PEGMA-950, and PEGMA-2000 described above). The calculated weight percentages of the methacrylic acid (MAA) and poly(ethylene glycol) (PEG) interpolymerized components in each of the grafted polymers of Functionalized Nonwovens A-S (FNWs A-S) were determined. In the calculations, the MAA monomers and poly(alkylene oxide) monomers (PAO monomers) were estimated to form grafted polymers having about the same proportion of monomer component by mass as the relative proportions of MAA and PAO monomers in the corresponding grafting solutions (procedures of Examples 1-10 and Example 14). The calculated values are reported in Table 11.

The weight percent (wt-%) of interpolymerized MAA in the grafted polymer was calculated using Equation 1.

$$wt ‐ \% MAA in grafted polymer = \frac{wt ‐ \% of MAA in grafting solution}{Total wt ‐ \% of monomers in grafting solution} \times 100 \% \qquad Equation 1$$

The weight percent (wt-%) of interpolymerized PEGMA monomer in the grafted polymer was calculated using Equation 2.

$$wt ‐ \% PEGMA in grafted polymer = \frac{wt ‐ \% PEGMA monomer in grafting solution}{Total wt ‐ \% of monomers in grafting solution} \times 100 \% \qquad Equation 2$$

The weight percent (wt-%) of the PEG component in the grafted polymer was calculated using Equation 3.

$$wt ‐ \% PEG in grafted polymer = ( Molar mass ratio of PEG in PEGMA monomer ) \times ( wt ‐ \% PEGMA in grafted polymer ) \qquad Equation 3$$

The Molar mass ratio of PEG in PEGMA monomer was calculated using Equation 4.

$$Molar mass ratio of PEG in PEGMA monomer = \frac{[ ( Molar mass of PEGMA monomer ) - ( Molar mass of all non ‐ PEG components of PEGMA monomer ) ]}{( Molar mass of PEGMA monomer )} \qquad Equation 4$$

For Equation 4, the non-PEG components of the PEGMA monomer are all portions of the PEGMA monomer excluding the repeat units of —CH₂—CH₂O—. For example, the methacrylate component (C₄O₂H₅, molecular mass=85 g/mol) and the terminal methyl group (CH₃, molecular mass=15 g/mol) are non-PEG components of a PEGMA monomer.

As an example for clarity, the calculated results for Functionalized Nonwoven N prepared using MAA (20 wt-%) and PEGMA-950 (5 wt-%) in the grafting solution are as follows:

$$\begin{matrix}wt ‐ \% MAA in grafted polymer = \frac{20 wt ‐ \% of MAA}{( 20 wt ‐ \% MAA + 5 wt ‐ \% PEGMA 950 )} \times 100 \% = 80 \% \\ wt ‐ \% PEGMA in grafted polymer = \frac{5 wt ‐ \% PEGMA 950}{( 20 wt ‐ \% MAA = 5 wt ‐ \% PEGMA 950 )} \times 100 \% = 20 \% \\ wt ‐ \% PEG in grafted polymer = ( 0.89 ) \times ( 20 wt \% PEGMA 950 in grafted polymer ) = 17.8 \%\end{matrix}$$

Molar mass ratio of PEG in PEGMA950 monomer=

$$\frac{[ ( 950 g / mol ) - ( 85 g / mol ) - ( 15 g / mol ) ]}{950 g / mol} = 0.89$$

In the ‘Molar mass ratio of PEG in PEGMA950’ equation, 950 g/mol is the molar mass of PEG950 monomer; 85 g/mol is the molar mass of the methacrylate component (C₄O₂H₅) of PEG950 monomer, and 15 g/mol is the molar mass of the methyl group (CH₃) component of PEG950 monomer.

TABLE 11

| | |
|---|---|
| | Calculated Weight Percent of the |
| | Interpolymerized MAA and PEG Monomer |
| Functionalized | Components of the Grafted Polymer |

| Nonwoven | wt-% MAA | wt-% PEG |
|---|---|---|
| FNW-A | 100% | 0% |
| FNW-B | 90.1% | 7.3% |
| FNW-C | 74.9% | 18.5% |
| FNW-D | 50.0% | 36.9% |
| FNW-E | 33.5% | 49.1% |
| FNW-F | 44.1% | 50.0% |
| FNW-G | 100% | 0% |
| FNW-H | 33.3% | 49.2% |
| FNW-I | 44.1% | 50.0% |
| FNW-J | 71.3% | 19.1% |
| FNW-K | 80.0% | 15.2% |
| FNW-L | 57.1% | 32.6% |
| FNW-M | 44.4% | 42.3% |
| FNW-N | 80.0% | 17.8% |
| FNW-O | 57.1% | 38.3% |
| FNW-P | 44.4% | 49.7% |
| FNW-Q | 80.0% | 19.0% |
| FNW-R | 57.1% | 40.7% |
| FNW-S | 44.4% | 52.8% |

The complete disclosures of the patents, patent documents, and publications cited herein are incorporated by reference in their entirety as if each were individually incorporated. To the extent that there is any conflict or discrepancy between this specification as written and the disclosure in any document that is incorporated by reference herein, this specification as written will control. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. Viral filtration media comprising:
a porous substrate comprising a surface having a polymer grafted thereto, wherein the grafted polymer comprises interpolymerized monomers comprising:
a (meth)acrylic acid monomer; and
a poly(alkylene oxide) monomer optionally including a hydrocarbon chain.

2. The filtration media of claim 1 wherein the (meth)acrylic acid monomer is methacrylic acid.

3. The filtration media of claim 1 wherein the poly(alkylene oxide) monomer has the formula:
wherein: 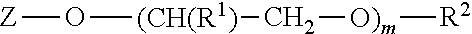
Z is a polymerizable ethylenically unsaturated group;
Q is a divalent linking group;
R¹ is H or an alkyl group;
R² is H or an alkyl group; and
m is 2 to 100.

4. The filtration media of claim 3 wherein Z is selected from the group consisting of: 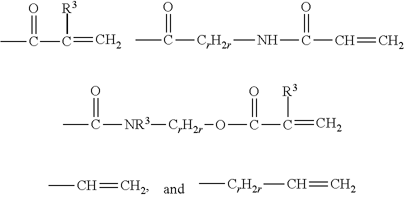
wherein:
R³ is H or CH₃; and
r is 1-10.

5. The filtration media of claim 3 where Q is selected from the group consisting of: —O—,
—NR³—, —C(O)O—, and —C(O)NR³—, wherein R³ is H or CH³.

6. The filtration media of claim 3 wherein R² is an alkyl group.

7. The filtration media of claim 3 wherein m is 2 to 50.

8. The filtration media of claim 1 wherein the poly(alkylene oxide) monomer is present in an amount of 2 wt-% to 75 wt-%, based on the total weight of the interpolymerized monomers.

9. The filtration media of claim 1 wherein the porous substrate is a porous polymeric nonwoven substrate.

10. An article comprising the viral filtration media of claim 1.

11. A method of filtering a virus-containing sample, the method comprising:
providing a viral filtration media comprising:
a porous substrate comprising a surface having a polymer grafted thereto,
wherein the grafted polymer comprises interpolymerized monomers comprising:
a (meth)acrylic acid monomer;
providing a virus-containing sample comprising a target virus; and
contacting the viral filtration media with the virus-containing sample under conditions effective to separate at least a portion of the target virus from other material in the virus-containing sample.

12. The method of claim 11 wherein the interpolymerized monomers comprise: a (meth)acrylic acid monomer; and a poly(alkylene oxide) monomer optionally including a hydrocarbon chain.

13. The method of claim 11 wherein contacting comprises allowing a moving virus-containing sample to impinge upon an upstream surface of the filtration media for a time sufficient to effect separation of at least a portion of the target virus from other material in the virus-containing sample.

14. The method of claim 13 wherein contacting comprises allowing a moving virus-containing sample to impinge upon an upstream surface of the filtration media for a time sufficient to effect binding of at least a portion of the target virus in the virus-containing sample to the filtration media.

15. The method of claim 13 wherein contacting comprises allowing a moving virus-containing sample to impinge upon an upstream surface of the filtration media for a time sufficient to effect binding of at least a portion of other material in the virus-containing sample to the filtration media.

16. The method of claim 13 wherein contacting comprises allowing a moving virus-containing sample to impinge upon an upstream surface of the filtration media for a time sufficient to effect separation of at least a portion of the target virus from another virus in the virus-containing sample.
